(19) United States
(12) Patent Application Publication
(10) Pub. No.: US 2023/0313150 A1
(43) Pub. Date: Oct. 5, 2023

(54) Bioluminscent Indicator and Sensor

(71) Applicant: Board of Trustees of Michigan State University, East Lansing, MI (US)

(72) Inventor: Eric Petersen, Lansing, MI (US)

(73) Assignee: Board of Trustees of Michigan State University, East Lansing, MI (US)

(21) Appl. No.: 18/153,170

(22) Filed: Jan. 11, 2023

Related U.S. Application Data
(60) Provisional application No. 63/102,004, filed on Jan. 11, 2022.

(51) Int. Cl. C12N 9/02; C12N 15/86; G01N 21/64; A61K 49/00

(52) U.S. Cl. CPC C12N 9/0069; A61K 49/0097; C12N 15/86; C12Y 113/12005; C12Y 113/12013; G01N 21/6428; C12N 2750/14143; G01N 2021/6439

(57) ABSTRACT

Disclosed herein are bioluminescent indictors comprising a bioluminescent peptide split by a binding peptide capable of binding a ligand. The bioluminescent indicator bioluminesces upon binding of the binding peptide to its ligand to bring the regions of the bioluminescent peptide into such proximity that the indicator bioluminesces. The bioluminescent indicator may further comprise a leader domain and a transmembrane domain. The bioluminescent indicator acts as a biosensor that can detect and quantify a ligand without the need for an additional light source. Methods for imaging internal body structures are also presented.

### CROSS-REFERENCE TO RELATED APPLICATIONS

This patent application claims the benefit under 35 U.S.C. § 119(e) to U.S. Provisional Application No. 63/102,004 filed on 11 Jan. 2022. The entire contents of 63/102,004 are hereby incorporated by reference.

### STATEMENT OF A SEQUENCE LISTING

The present disclosure contains references to amino acid sequences and nucleic acid sequences which have been submitted concurrently herewith as the sequence listing text file entitled “000426us_SequenceListing.xml,” file size 63.1 KiloBytes (KB), created on 10 Jan. 2023. The aforementioned sequence listing is hereby incorporated by reference in its entirety pursuant to 37 C.F.R. § 1.52(e)(5).

### GOVERNMENTAL RIGHTS

This invention was made with government support under 2027713, awarded by the NSF; under NS098231 and NS104306, awarded by NIH/NINDS; under EB031008, EB031936, and EB024495 awarded by NIH/NIBIB. The government has certain rights in the invention

### FIELD

The present disclosure relates to a bioluminescent indicator comprising a binding domain incorporated between two bioluminescent domains, wherein binding of a desired ligand induces a conformational change to bring the two bioluminescent domains together for bioluminescence.

### BACKGROUND

Optical biosensors have proven incredibly useful to researchers in a wide variety of fields for the study of neuronal and cellular activity. These probes generate changes in light emission intensity and/or wavelength in response to physiological events such as calcium influx, membrane voltage changes, or the presence of a ligand such as a neurotransmitter. Currently, nearly all available biosensors rely on fluorescence, leaving much room for improvement and further development of new approaches to better report changes in these cellular dynamics. One major improvement to such indicators would be to eliminate the need for an excitation light source, which is necessary to excite fluorescent reporters.

The illumination source used for fluorescent imaging is a major limiting factor for the depth at which cells can be imaged through tissue. This is due to the scattering of light traveling into the tissue, as well as heating as the incident photons are absorbed by endogenous molecules in the tissue. Imaging with bioluminescent probes eliminates the need for excitation light, circumventing these issues and enabling researchers to image deeper structures of the brain³. Furthermore, autofluorescence produced by the illumination sources used for fluorescent imaging is not present when using bioluminescence, allowing for enhanced signal detection in deeper structures since the signal to noise ratio can be higher. These fundamental limitations to fluorescence imaging and corresponding benefits of bioluminescence imaging dovetail with an urgent need in the field of neuroscience: tools that allow recording and modulation of entire neuronal populations that are both non-invasive and don’t require implanted hardware. (FIG. 1).

Bioluminescence is produced by an enzyme (e.g., luciferase) that catalyzes oxidation of its specific substrate (such as luciferin is for the luciferase enzyme), resulting in the emission of light. Different forms of biological light production exist in multiple domains in nature, including beetles, worms, bacteria, and the majority of marine organisms. Bioluminescence has been used for a variety of imaging applications, such as the quantification of gene expression over time and for imaging calcium dynamics to represent neuronal function or to track other calcium events within cells. Although bioluminescence has been used in research for decades, more recently scientists are rapidly improving luciferases and synthetic luciferins. For example, a 1000-fold increase in luminescence was recently achieved by evolving firefly luciferase to use a synthetic substrate to create AkaLuc which produces near infrared bioluminescence. See, for example, Iwano, S. et al. Science 359, 935-939 (2018), which is incorporated herein by reference.

Before genetically-encoded indicators were available, the standard method to analyze specific neurotransmitters in vivo was to collect cerebrospinal fluid from within the brain via microdialysis or cyclic voltammetry, both of which offer poor time resolution (multiple minutes) and have decreasing performance in longitudinal studies. In the past several years, fluorescent genetically encoded neurotransmitter indicators such as iGluSnFr, Dlight and GRAB-DA have enabled detection of neurotransmitters on a faster time scale. Unfortunately, fiber photometry, the implantation of an optical fiber into the brain, is required to measure the output of these fluorescent probes in deep regions of the brain

### SUMMARY

Disclosed herein is a bioluminescent indicator comprising a first domain, a second domain, and a third domain. The first domain comprises a first portion of a bioluminescent peptide, the second domain comprises a binding peptide, and the third domain comprises a second portion of the bioluminescent peptide. The bioluminescent indicator is configured such that when the binding peptide undergoes a confirmational change upon binding to its ligand, it brings the first and third domains into such proximity to induce bioluminescence. In some embodiments, the bioluminescent indicator further comprises a leader domain, a transmembrane domain, and/or linking sequences between the first and second domains and/or between the second and third domains.

Also described herein are nucleotide sequences encoding the bioluminescent indicator. The nucleotide sequences may be a part of a vector, such as an adeno-associated virus.

Also described herein are methods for detecting the presence or absence of a compound (e.g., ligand) in a sample or a subject, the method comprising providing the bioluminescent indicator or a nucleotide sequence encoding the bioluminescent indicator to the subject or sample, and detecting an amount of bioluminescence of the bioluminescent indicator.

Various objects, features, aspects, and advantages will become more apparent from the following detailed description of preferred embodiments, along with the accompanying drawings.

### BRIEF DESCRIPTION OF THE DRAWINGS

FIG. 1A depicts a schematic of the BioLuminescent Indicator of the Neurotransmitter Glutamate (BLING) with a split luciferase (dark grey) and a glutamate sensing peptide (light grey) displayed on a cell surface. FIG. 1B depicts a comparison of bioluminescence and fluorescence imaging for superficial and deep tissue targets.

FIG. 2A depicts a protein map of BLING 0.1, BLING 0.2, and BLING 0.3. FIG. 2B depicts the results of the BLING variants in response to 1 mM glutamate in HEK cells.

FIG. 3A depicts a protein map of BLING 1.0. FIG. 3B depicts the luminescence in response to glutamate from the BLING variant library with variable linkers.

FIG. 4A depicts the response of BLING 1.0 to 1 mM glutamate. FIG. 4B depicts the responses of bioluminescent indicators BLING 0.2 and BLING 1.0 or fluorescent indicators iGluSnFr and GCaMP6m in cell culture. FIG. 4C depicts the dose dependent response of BLING 1.0 to increasing doses of glutamate.

FIG. 5A depicts an example trace of a single cell ROI showing perfusion of furimazine and infusion of 1 mM glutamate. FIG. 5B depicts the dose response of BLING 0.2 and BLING 1.0 to increasing doses of glutamate on a single cell (n = 4) *=p<0.05. FIG. 5C depicts an image of background bioluminescence of BLING 1.0 (0 µM glutamate) and BLING 1.0 luminescence in the presence of 1 mM glutamate.

FIG. 6A shows a schematic of the in vivo experimental design for expression of BLING 1.0 in the sensory cortex of a rat. FIG. 6B depicts exemplary images of bioluminescence before and after seizure induction with bicuculline. FIG. 6C depicts an exemplary trace of bioluminescent intensity in response to acute seizure induction with bicuculline.

FIG. 7A presents a schematic of the bioluminescent indicator comprising a dopamine binding peptide in the second domain. FIG. 7B depicts the luminescence in response to dopamine from the DLume variant library with variable linkers. FIG. 7C depicts the results of the DLume variants in response to 1 mM dopamine in HEK cells.

FIG. 8A presents a schematic of the bioluminescent indicator comprising a voltage sensing domain. FIG. 8B depicts the luminescence in response to voltage changes from the VoLume variant library with variable linkers. FIG. 8C depicts the results of the VoLume variants in response to changes in voltage in HEK cells.

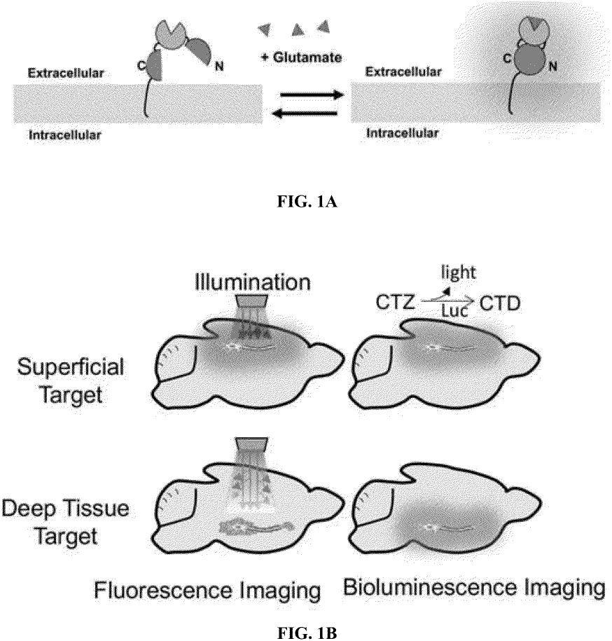

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell,” for example, includes a plurality of the cells of the same type. Furthermore, to the extent that the terms “including,” “includes,” “having,” “has,” “with,” or variants thereof are used in either the detailed description and/or the claims, such terms are intended to be inclusive in a manner similar to the term “comprising.”

As used in this specification and the appended claims, “or” is generally employed in its sense including “and/or” unless the content clearly dictates otherwise.

“About” as used herein when referring to a measurable value such as an amount, a temporal duration, and the like, is meant to encompass variations that would round out from a value past the last significant digit. For example, the designation “about 2.5” reads a range of values from 2.45 (which would round up to 2.5) to 2.54 (which would round down to 2.5).

The term “disease” is meant any condition or disorder that damages or interferes with the normal function of a cell, tissue, or organ. Examples of diseases that can be treated with the compositions disclosed herein include neoplasia, cancer, neurodegenerative or neurologic diseases (e.g., spinal cord injury, Parkinson’s disease, Alzheimer’s disease), pain (e.g., neuropathic pain, nociceptive pain, inflammatory pain), autoimmune diseases, viral infections, and senescent cell-and age-related diseases.

“Effective amount” refers to the amount and/or dosage, and/or dosage regime of one a composition comprising the bioluminescent indicator described herein, or a nucleotide sequence encoding the bioluminescent indicator, described herein necessary to bring about the desired result e.g., an amount sufficient bind detect a ligand in a subject or a sample.

“Identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity for purposes of the present disclosure is determined using the BLAST algorithm, described in Altschul & al. (199) J. Mol. Biol. 215:403-10, which is incorporated herein by reference. The BLAST algorithm is publicly available through software provided by the National Center for Biotechnology Information (at the web address www.ncbi.nlm.nih.gov). This algorithm identifies high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul & al., supra.). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence or nucleic acid sequence, the default parameters of the BLAST programs can be used. For analysis of amino acid sequences, the BLASTP defaults are: word length (W), 3; expectation (E), 10; and the BLOSUM62 scoring matrix. For analysis of nucleic acid sequences, the BLASTN program defaults are word length (W), 11; expectation (E), 10; M=5; N=-4; and a comparison of both strands. The TBLASTN program (using a protein sequence to query nucleotide sequence databases) uses as defaults a word length (W) of 3, an expectation (E) of 10, and a BLOSUM 62 scoring matrix. (see Henikoff & Henikoff (1989) Proc. Natl. Acad. Sci. USA 89:10915, which is incorporated herein by reference).

In addition to calculating percent sequence identity, the BLAST algorithm also performs a statistical analysis of the similarity between two sequences (see, e.g., Karlin & Altschul (1993) Proc. Nat′l. Acad. Sci. USA 90:5873-87, which is incorporated herein by reference). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01.

“Parenteral” administration of an immunogenic composition includes, e.g., subcutaneous (s.c.), intravenous (i.v.), intramuscular (i.m.), or intrasternal injection, or infusion techniques.

“Patient” or “individual” or “subject” are used interchangeably herein, and refers to a mammalian subject to be treated, interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker.

“Sample” refers to cells, explants, biopsy, or tissues, either in vivo or ex vivo, from a human or animal described above. The sample may be obtained by direction from a physician or health care worker. Alternatively, the sample may include extracellular or acellular material, including but not limited to cell culture media, lysed cells, urine, whole blood, plasma, serum, lymph, mucus, expressed breast milk, semen, stool, sputum, cerebral spinal fluid, tears, hair, and saliva. Additionally, the sample may be an environmental sample.

A “pharmaceutically acceptable” component/carrier etc. is one that is suitable for use with humans and/or animals without undue adverse side effects (such as toxicity, irritation, and allergic response) commensurate with a reasonable benefit/risk ratio.

“Specifically binds” means that a compound or antibody recognizes and binds a polypeptide of the invention, but which does not substantially recognize and bind other molecules in a sample, for example, a biological sample, which may include a polypeptide of the invention.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Genbank and NCBI submissions indicated by accession number cited herein are incorporated herein by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### Bioluminescent Indicator

In general, the bioluminescent indicator may be a protein, such as a fusion protein, comprising a first domain, a second domain, and a third domain. The first and third domains, when brought together represent a single bioluminescent peptide that bioluminesces only when the first and third domains are in proximity. The second domain represents a binding peptide which binds a ligand. The ligand binding induces a conformational change in the second domain which brings the first and third domains into such proximity to allow the bioluminescent indicator to bioluminescence. In some embodiments, the second domain is located between the first and third domains.

The second domain may be placed between the first and third domains. Thus, the first and third domains each comprise a portion of the bioluminescent peptide. Together, the first and third domains represent a sufficient portion of the bioluminescent peptide that remains capable of bioluminescence when close to each other. The first and third domains may comprise an equal or near equal number of amino acids in each domain, or they may comprise an unequal number of amino acids in each domain. The second domain may be placed between the first and third domains at a location such that the first and third domains do not bioluminesce or display substantially reduced bioluminescence as compared to the corresponding bioluminescent peptide as a whole when bound to its target.

The identity of the bioluminescent peptide divided across the first and third domains is not particularly limited so long as each portion separately does not itself possess bioluminescence but does bioluminesce when brought in proximity to each other. In some embodiments, the bioluminescent peptide may be luciferase. The luciferase may include naturally occurring, modified, or synthetic luciferases, including but not limited to firefly luciferase, bacterial luciferases, fungal luciferases, and marine luciferases (e.g., luciferase from one of Metridia, Oplophorus gracilirostis, Gaussia princeps, Renilla reniformis, and the like). Also included include modified variants thereof. In a particular embodiment, the bioluminescent peptide divided across the first and third domains may be luciferase from Gaussia princeps, luciferase from Renilla reniformis, or a synthetic luciferase, such as NanoLuc® (Promega).

The identity of the binding peptide of the second domain is not particularly limited. The binding peptide may bind its cognate ligand, which induces a confirmational change in the three-dimensional structure of the binding peptide. The binding peptide has sufficient affinity and specificity for its ligand such that the peptide can distinguish between its cognate ligand and an irrelevant ligand. By way of non-limiting examples, the binding of the second domain may bind to dopamine or glutamine. The binding peptide has sufficient specificity such that if it is a binding peptide for dopamine, it will not bind another ligand, for example glutamate. Alternatively, if the binding peptide does bind another ligand, such binding does not induce a confirmational change.

Additionally, the bioluminescent indicator may further comprise a leader domain. The leader domain may be useful for cell surface display. The leader domain is not particularly limited and may be any sequence that the allows for cell surface display or direction within the cell to another specified location. The leader domain may be Ig kappa (Igx), Ig heavy, oncostatin M (OSM), VSV-G, BM40, tPA, HSA albumin, and/or influenza hemagglutinin. In a preferred embodiment, the leader domain comprises Igκ, or a sequence having at least about 75% sequence identity, at least about 80% sequence identity, at least about 85% sequence identity, at least about 90% sequence identity, or at least about 95% sequence identity to Igκ.

Additionally or alternatively, the bioluminescent indicator may further comprise a transmembrane domain. The transmembrane domain serves to anchor the bioluminescent indicator in a cell membrane. The transmembrane domain is not particularly limited and may be any sequence that adequately anchors the bioluminescent indicator in the cell membrane, or other membrane. In a particular embodiment, the transmembrane domain may be platelet-derived growth factor receptor β (PDGFRβ), or a sequence having at least about 75% sequence identity, at least about 80% sequence identity, at least about 85% sequence identity, at least about 90% sequence identity, or at least about 95% sequence identity to PDGFRβ.

Additionally or alternatively, the bioluminescent indicator may further comprise a linker between the first and second domains, between the second and third domains, or both between the first and second domains and between the second and third domains. The linker may be flexible or rigid. The linker may have a sequence of between 3 and 20 amino acids, such as 3 amino acids, 5 amino acids, 10 amino acids, 15 amino acids, and 20 amino acids or all integers therebetween. In a particular embodiment, the linker may be a glycine-rich linker (e.g., GGGGS (SEQ ID NO: 9)), a proline-alanine linker (e.g., PAPAP (SEQ ID NO: 10)), or a variable 3-amino acid sequenced composed of a combination of alanine, serine, and proline (ASP) sequence.

The components of the bioluminescent indicator may be in a sequence of leader domain-first domain-linker-second domain-linker-third domain-transmembrane domain. Particular embodiments of the bioluminescent indicator of glutamate (e.g., “BioLuminescent Indicator of Neurotransmitter Glutamate” or “BLING”) are show in Table 1 below.

TABLE 1

| Bioluminescent Indicator | SEQ ID NO (AA) | SEQ ID NO (DNA) |
|---|---|---|
| BLING 0.1 | 1 | 5 |
| BLING 0.2 | 2 | 6 |
| BLING 0.3 | 3 | 7 |
| BLING 1.0 | 4 | 8 |

In some embodiments, the bioluminescent indicator may have an amino acid sequence having at least about 75% sequence identity, at least about 80% sequence identity, at least about 85% sequence identity, at least about 90% sequence identity, or at least about 95% sequence identity of SEQ ID NO: 5, SEQ ID NO: 6, SEQ ID NO: 7, or SEQ ID NO: 8. Additionally or alternatively, the bioluminescent indicator may have an amino acid sequence comprising the sequence SEQ ID NO: 5, SEQ ID NO: 6, SEQ ID NO: 7, or SEQ ID NO: 8. In a further embodiment, the bioluminescent indicator may comprise an amino acid sequence encoded by a nucleotide sequence having at least about 75% sequence identity, at least about 80%, at least about 85% sequence identity, at least about 90% sequence identity, or at least about 95% sequence identity with a sequence of SEQ ID NO: 1, SEQ ID NO: 2, SEQ ID NO: 3, or SEQ ID NO: 4. Additionally or alternatively, the bioluminescent indicator may comprise an amino acid sequence encoded by a nucleotide sequence of SEQ ID NO: 1, SEQ ID NO: 2, SEQ ID NO: 3, or SEQ ID NO: 4.

### Nucleic Acid Sequences and Vectors

The bioluminescent indicator described above can be prepared by conventional means known in the art. The amino acid sequences may be encoded by a nucleotide sequence (e.g., a DNA sequence) and provided to a cell (e.g., a hybridoma, bacteria, yeast, etc.) which translates the nucleotide sequence to the protein. The nucleotide sequences described herein may be incorporated into a vector (e.g., a viral vector, a plasmid, etc.) for insertion into a cell (e.g., transformation, transfection, etc.) for subsequent production of the bioluminescent indicator.

The nucleotide sequence may comprise a DNA sequence encoding the first domain, second domain, and/or the third domain as described above. For example, the nucleotide sequence may encode a first and/or third domain, where the first and third domain are each a first and second portion of a bioluminescent peptide (e.g., luciferase). The nucleotide sequence further encodes a second domain comprising a binding peptide inserted between the first and third domains. The nucleotide sequence may further include a sequence encoding a leader domain (e.g., an Igκ leader sequence) and/or a transmembrane domain (e.g., PDGFRβ).

In any embodiment, a nucleotide sequence encoding any of the bioluminescent indicator is provided. For example, a nucleotide sequence may encode BLING 0.1, BLING 0.2, BLING 0.3, or BLING 1.0. The nucleotide sequence may have a nucleotide sequence having at least about 75% sequence identity, at least about 80%, at least about 85% sequence identity, at least about 90% sequence identity, or at least about 95% sequence identity with a sequence of SEQ ID NO: 1, SEQ ID NO: 2, SEQ ID NO: 3, or SEQ ID NO: 4. Additionally or alternatively, the nucleotide sequence may comprise SEQ ID NO: 1, SEQ ID NO: 2, SEQ ID NO: 3, or SEQ ID NO: 4.

In certain embodiments, the polynucleotides described above can be expressed in a supporter cell line. Mammalian cell lines such as human embryonic kidney (HEK) cells, Chinese hamster ovary (CHO) cells, or 293T cells are particularly suitable for these purposes. The proteins described above may be generally soluble and may be excreted from a producing cell unless they are modified for intracellular retention. Proteins produced in this manner can be purified from the culture medium. Where desired, the proteins may be tagged with (e.g.) a poly-histidine tag or other such commercially common tags to facilitate purification. Proteins produced and purified in this manner can then be administered to a subject in need thereof as described below.

Additionally or alternatively, the polynucleotides described above can be incorporated into a vector (e.g., a transfection vector or a viral transduction vector). Such vectors can then be transfected or transduced into a subject’s cells or in cells cultured in vitro. In this way, the subject’s own cells (e.g., the subject’s brain tissue) may produce bioluminescent indicators described above.

A variety of expression systems exist for the expression of the bioluminescent indicator in a cell or tissue of interest. Expression vectors useful for producing such polypeptides include, without limitation, chromosomal, episomal, and virus-derived vectors. Vectors include, but are not limited to plasmids, bacteriophage, transposons, yeast episomes, insertion elements, yeast chromosomal elements, and viruses. Specific viral vectors include, but are not limited to baculoviruses, papova viruses (e.g., such as SV40), vaccinia viruses, lentiviruses, adenoviruses, adeno-associated viruses (AAV), fowl pox viruses, pseudorabies viruses, and retroviruses, and vectors derived from combinations thereof.

In a specific embodiment, the vector includes a nucleotide sequence encoding a DNA sequence encoding the bioluminescent indicator described above. The vector may comprise a DNA sequence having at least about 75% sequence identity, at least about 80%, at least about 85% sequence identity, at least about 90% sequence identity, or at least about 95% sequence identity to one of SEQ ID NO: 1, SEQ ID NO: 2, SEQ ID NO: 3, or SEQ ID NO: 4. Alternatively, the vector comprises a DNA sequence comprising one of SEQ ID NO: 1, SEQ ID NO: 2, SEQ ID NO: 3, or SEQ ID NO: 4. The vector comprising the DNA sequence may be an adeno-associated virus.

The vectors described here may also include a regulatory region. The term “regulatory region” may refer to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to the positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

### Pharmaceutical Therapeutics

The bioluminescent indicators, nucleotide sequences encoding the bioluminescent indicators, or vectors comprising said nucleotide sequences may be provided in pharmaceutical compositions for use as a therapeutic agent. The bioluminescent indicators, nucleotide sequences encoding the bioluminescent indicators, or vectors may be formulated with a pharmaceutically acceptable carrier to prepare the pharmaceutical composition for use to treat, for example, neoplasia, cancer, neurodegenerative or neurologic diseases (e.g., spinal cord injury, Parkinson’s disease, Alzheimer’s disease), pain (e.g., neuropathic pain, nociceptive pain, inflammatory pain), autoimmune diseases, viral infections, and senescent cell-and age-related diseases.

The bioluminescent indicators described herein can be used to control light sensitive molecules, including but not limited to light sensitive ion channels and light sensitive transcription factors. Controlling the light with the bioluminescent indicators in close proximity to the light sensitive molecules within a cell or tissue (e.g., neurons) can induce a therapeutic effect. In such instances, the bioluminescent indicator may produce light in the presence of a neurotransmitter of interest (e.g., glutamate or dopamine) to activate or inhibit the light sensitive molecule to control neuronal activity. In some embodiments, the bioluminescent indicator may be used to induce synaptic plasticity in an activity-dependent manner or inhibit neural transmission within an affected system. In another embodiment, the bioluminescent indicators described herein may control or otherwise influence cell metabolism (e.g., metabolism based on intracellular levels of glutamate). A binding peptide of the second domain may bind to a transcription factor, ion channel, cytokine, or other molecule in a cellular pathway and, in so doing, the bioluminescent indicator can be specifically targeted to a particular cell, tissue, tumor, or cancer. Upon binding to the binding peptide of the second domain, the first and second portions of the bioluminescent peptide of the first and third domains are brought into close proximity to generate light. The generated light would then influence the light sensitive molecule within the cell our in an extracellular space.

### Pharmaceutical Diagnostics

The bioluminescent indicators, nucleotide sequences encoding the bioluminescent indicators, or vectors comprising said nucleotide sequences may be provided in pharmaceutical compositions for use as a diagnostic agent. The bioluminescent indicators, nucleotide sequences encoding the bioluminescent indicators, or vectors may be formulated with a pharmaceutically acceptable carrier to prepare the pharmaceutical composition. The pharmaceutical composition may be administered systemically, for example, formulated in a pharmaceutically acceptable buffer such as physiological saline. Preferable routes of administration include, for example, instillation into the bladder, subcutaneous, intrathecal, intracranial, intravenous, intraperitoneal, intramuscular, intratumoral, or intradermal injections that provide continuous, sustained, or effective levels of the composition in the patient. Alternatively the pharmaceutical composition may be administered locally, such as administered directly to a particular tissue, such as a diseased tissue or a healthy (normal) tissue. In particular, the bioluminescent indicator and pharmaceutically acceptable carrier may be formulated for parenteral injection, including but not limited to subcutaneous, intravenous, intramuscular, intravesicular, intratumoral, or intraperitoneal injection.

The pharmaceutical compositions comprising a bioluminescent indicator or nucleotide sequence encoding the bioluminescent indicator may be in a form suitable for sterile injection. To prepare such a composition, the bioluminescent indicator or a nucleotide sequence encoding the bioluminescent indicator may be dissolved or suspended in an acceptable vehicle, including, but not limited to, water, water adjusted to a suitable pH (e.g., by addition of an appropriate amount of hydrochloric acid, sodium hydroxide or a suitable buffer), 1,3-butanediol, Ringer’s solution, isotonic sodium chloride solution, and/or dextrose solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added.

Additionally or alternatively, the bioluminescent indicator, or nucleotide sequence encoding the same, may be used in a method to detect the presence or absence of a ligand or agent (e.g., glutamate or dopamine), wherein a composition comprising the bioluminescent indicator, or nucleotide sequence encoding the same, is provided to the subject or sample and the luminescence of the indicator is measured. The bioluminescent indicator or nucleotide sequence encoding the same are administered in an amount effective for diagnosis. The bioluminescent indicator may be provided directedly to the desired tissue (e.g., via a direct local injection of the bioluminescent indicator). Alternatively, the bioluminescent indicator may be provided to the subject via administration of a vector (e.g., an adeno-associated virus vector) encoding the bioluminescent indicator. The method includes the step of administering to the subject an effective amount of a compound herein sufficient to diagnose a disease or disorder or symptom thereof.

The methods described herein provide for determining the presence of a particular ligand or agent which binds to the binding peptide of the second domain (e.g., glutamate or dopamine). When the binding peptide of the second domain recognizes and binds to its ligand, the binding peptide undergoes a conformational change to bring the first and third domains of the bioluminescence indicator into sufficient proximity to induce luminescence that can be read and measured.

### Pharmaceutical Systems

Kits or Diagnostic compositions comprising the bioluminescent indicator, or nucleotide sequences encoding the same, may be assembled into kits or pharmaceutical systems for use in detecting the presence or absence of a ligand in a sample or subject. Kits or diagnostic systems according to this aspect of the invention comprise a carrier means, such as a box, carton, tube, having in close confinement therein one or more container means, such as vials, tubes, ampoules, bottles, and the like. The kits or pharmaceutical systems of the invention may also comprise associated instructions for using the bioluminescent indicator and/or nucleotide sequences encoding the same described above.

### EXAMPLES

### Example 1 — Sensor Design

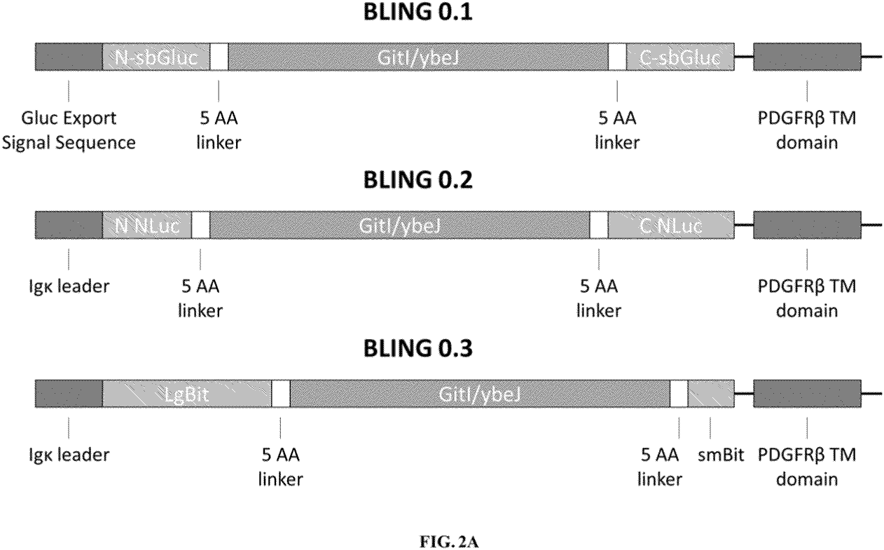

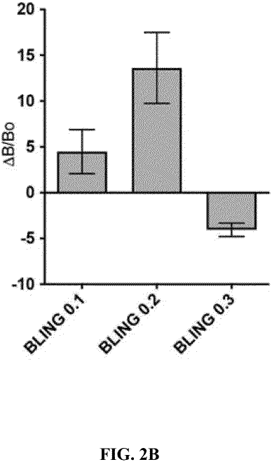

Three bioluminescent indicator variants (BLING 0.1, BLING 0.2, and BLING 0.3 (FIG. 2A) were constructed using Glt1, a truncated periplasmic glutamine binding peptide (second domain), an Igκ leader sequence, and a PDGFRβ transmembrane sequence (GenBank EU42295) synthesized as a Gblock or oligos used for PCR and assembled into a pcDNA 3.1 vector using Gibson Assembly (Neb HiFi) with their respective luciferase fragments (described below). The glutamine binding peptide were flanked with short flexible, five amino acid linkers. BLING 0.1 comprises a luciferase M43L, M110L variant from Gaussia princeps, a luciferase, that is split between amino acid positions 105 and 106, including the 17 AA native secretion signal, as the first and third domains. BLING 0.2 comprises NanoLuc®, a synthetic luciferase split between amino acids 66 and 67, with an Igκ leader sequence for cell surface display. BLING 0.3 comprises NanoLuc® large and small portions, split between amino acids 159-160.

HEK cells were plated on Poly-D-Lysine coated white 96 well plates and grown to 50-70% confluency. The HEK cells were transfected with 0.5 mL Lipofectamine 2000® per mL Opti MEM with 100 ng DNA per well using 20 µL of the transfection mix per well. Initial testing was performed with 5 mM native CTZ for BLING 0.1 or 5 mM hCTZ for BLING 0.2 and BLING 0.3 (Nanolight Technologies #301) in FluoroBrite media (Thermo). The media was changed 15 minutes prior to reading to allow the reaction to stabilize. The plates were read using a BioTek Cytation 5, by injecting 10 µL of 20 mM glutamate stock into 190 µL media for a final concentration of 1 mM. The luminescence of BLING 0.1, BLING 0.2, and BLING 0.3 in response to CTZ or hCTZ are shown in FIG. 2B.

### Example 2 — Library Construction and Screening

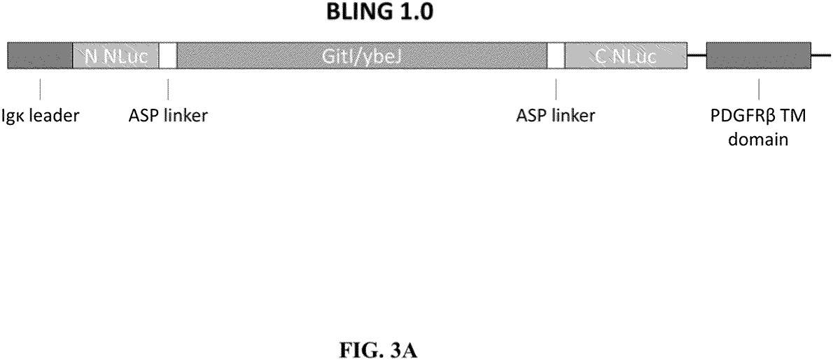

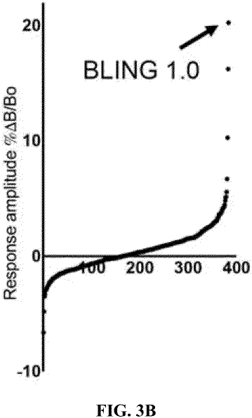

BLING 0.2 was selected for further improvement. The linker sequences flanking the binding peptide of the second domain were converted to various 3-amino acid linkers comprising one of alanine, serine, or proline (ASP linker) at each location of the linker. The BLING 0.2 variants were otherwise identical to BLING 0.2, comprising NanoLuc® (a synthetic luciferase split between amino acids 66 and 67), an Igκ leader domain and a PDGFRβ transmembrane domain. Assembly products were digested with DpnI to eliminate any plasmid template material to eliminate background colonies and electroporated into Top10 cells (Thermo). Colonies were then grown in deep 96-well plates in 1.5 mL LB media overnight and miniprepped in 96-well format (Biobasic #B814152-0005). Each plasmid was transfected into HEK cells grown in Poly-D-Lysine coated white 384-well plates in quadruplicate to generate an average for each variant tested. A transfection master mix was prepared with 21 mL Opti MEM with 100 µL Lipofectamine 2000®, distributed into four 96-well PCR plates, 50 µL per well. 5 µL DNA from the 96-well mini preps was added to the transfection master mix with mini prep DNA yields ranging from 100-200 ng/µL. Testing was performed two days later using 5 µM hCTZ in FluoroBrite media. Media was changed 15 minutes prior to reading. The plates were read using a BioTek Cytation 5, injecting 5 µL of 20 mM glutamate stock into 95 µL media for a final concentration of 1 mM. The BLING 0.2 variants were tested as above and the luminescence was determined. (FIG. 3B) The top performing BLING 0.2 variant is termed BLING 1.0 (FIG. 3A) and investigated for further characterization.

BLING 1.0 was compared to BLING 0.2. HEK cells were plated on white 96-well plates, grown to 50-70% confluency, and transfected with 0.5 µL Lipofectamine 2000® per mL Opti MEM with 100 ng DNA per well using 20 µL of the transfection mix per well. Measurements were taken with 5 µM hCTZ in FluoroBrite media, changed 15 minutes prior to reading on a Tecan spark for bioluminescence, GCaMP6m and iGluSnFr (fluorescent agents) were taken with a Biotek Cytation 5 using 1 mM glutamate or 5 µM ionomycin, respectively. The concentration dependent experiment was done in HBSS with magnesium and calcium in 10 mM HEPES buffer and 1 µM hCTZ. Statistical analysis was performed using a one-way ANOVA or two-way repeated measures with Bonferroni post-hoc n=2-3 per group.

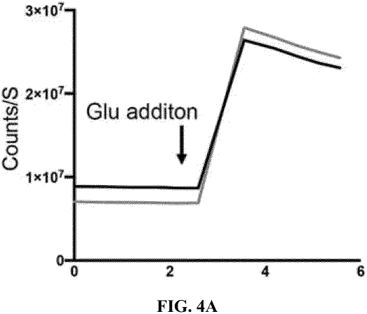

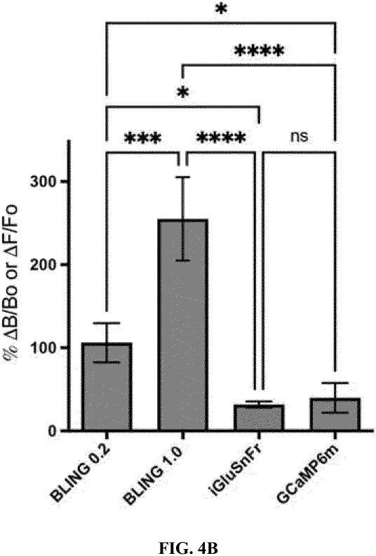

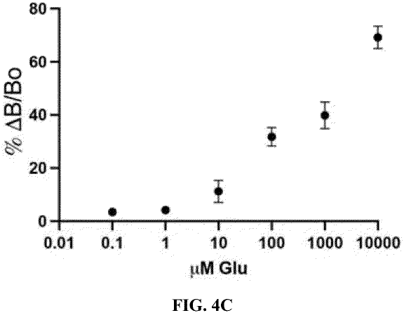

BLING 1.0 shows a robust response to glutamate addition in mammalian cells. (FIG. 4A). BLING 1.0 outperforms BLING 0.2 by over 2-fold in response to glutamate addition while maintaining brightness across multiple plate reading modalities. BLING 0.2 and BLING 1.0 (both of which are luminescent) outperform fluorescent markers iGLuSnFr and GCaMP6m (which require a light input for light emission). (FIG. 4B) BLING 1.0 shows as increase in bioluminescence directly related to glutamate concentration, which also showing minimal background levels of luminescence in the absence of glutamate. (FIG. 4C)

### Example 3 — Microscopy

For microscopy experiments, HEK cells were seeded in 12-well plates at 8x10⁵ per well and transfected the following day using 4 µL Lipofectamine 2000® in 100 mL Opti MEM and 2 µg DNA in 100 mL Opti MEM. The transfected cells were incubated overnight, trypsinized (TrypLE, Thermo), and plated on Poly-D-Lysine coated 18 mm cover slips (NeuVitro). The cells were imaged one to three days later. Imaging was performed with a Zeiss Al Axioscope, 5x 0.17 NA objective, Andor iXon 888 EMCCD camera, EM gain of 600, 4x4 binning with an open optical path and microscope within a dark box. Imaging was done in a perfusion chamber with artificial cerebral spinal fluid (ACSF) using 1 µM furimazine (Promega), heated to 37° C. ACSF was continually perfused followed by ACSF with furimazine followed by ACSF with furimazine and the respective concentration of glutamate followed by a washout with ACSF. The same ROI was used for all concentrations, statistical analysis done using a repeated measures 2-way ANOVA with Bonferroni post-hoc, n=8 per group. Images were analyzed using ImageJ for background subtraction and despeckling to reduce noise, ROIs selected manually for quantification.

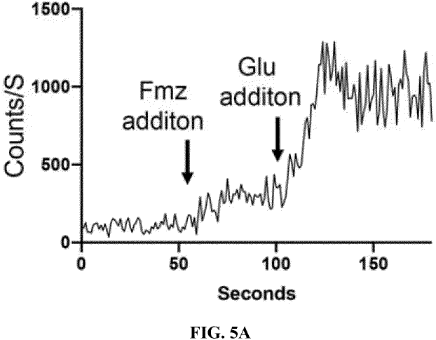

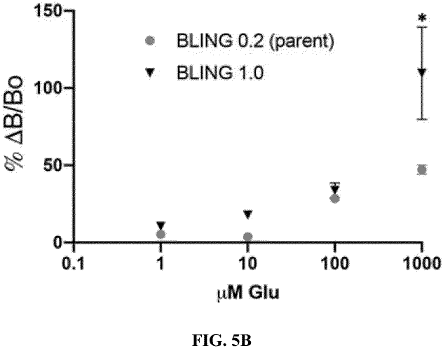

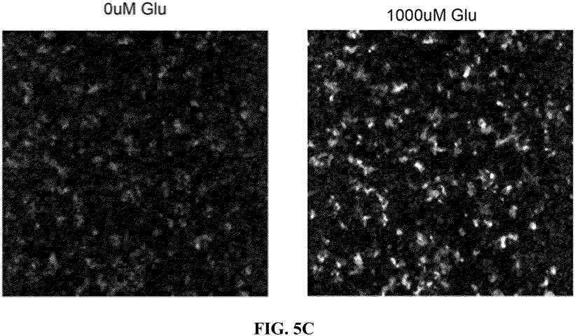

BLING 0.2 and 1.0 successfully report changes in extracellular glutamate levels at a single cell level. HEK cells expressing BLING 1.0 shows a 310% in bioluminescence at the single level following addition of glutamate up to 1 mM. (FIGS. 5A and 5C) BLING 1.0 appears sensitive to physiological levels of glutamate as compared to BLING 0.2. (FIG. 5B)

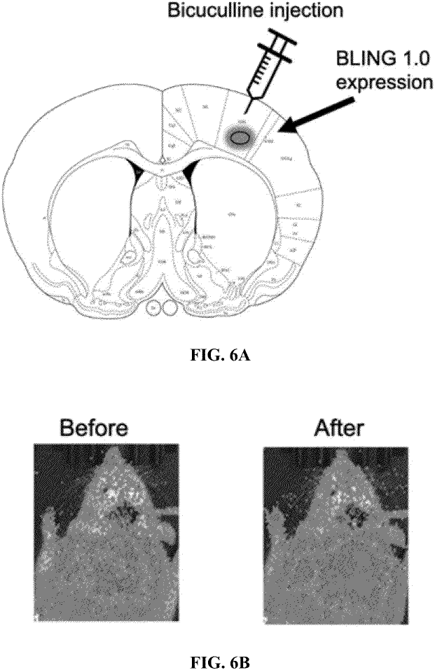

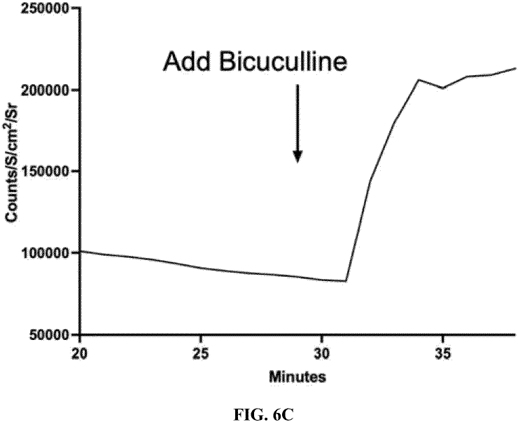

Example 4 — in vivo glutamate detection: BLING 1.0 was cloned into an adeno-associated virus (AAV) expression vector with a synapsin promotor for expression in neurons. AAV was prepared as described in Petersen, BioRxiv, 710194 (2019), which is incorporated herein by reference in its entirety. Sprague Dawley rats were injected with 2 µL of of 0.5x10¹² copies/mL at rate of 0.5 µL per minute with a 33G world precision syringe at 0 mm AP 3.5 mm R, and 2 mm ventral of the cortical surface. The needle was left in place for 5 minutes after infusion and slowly retracted. Imaging was done at least three weeks later with an IVIS spectrum (Perkin Elmer). Animals were injected with water soluble in vivo h-CTZ (Nanolight Technologies) 2 mg/kg intraperitoneally one to two hours prior to imaging and checked for the onset of bioluminescence. Once bioluminescence was detected, the incision was reopened, and a custom-made cannula was inserted through the small burr hole from the prior surgery for bicuculline administration. (FIG. 6A) Then a baseline recording of ten minutes was acquired and 10 µL of 10 mM bicuculline was slowly injected over five minutes. Images were captured using large binning, fstop of one with one-minute exposures. Images were analyzed with Living Image software with an ROI over the entire skull. All animal work was approved by the Michigan State Universities Institutional Animal Care and Use Committee.

BLING 1.0 was expressed with AAV in the sensory cortex 2 mm below the surface of the skull. The region injected with the BLING 1.0-containing AAV show expression of BLING 1.0. (FIG. 6B) Luminescence in response to glutamate binding to BLING 1.0 increased approximately 2-fold following bicuculline administration. (FIG. 6C)

### Example 4 — Alternate Bioluminescent Variants

BLING variants utilizing other luciferases were prepared using the techniques above. The variants utilize luciferases including Gaussia luciferase, Renilla luciferase, and alternate split sites in NanoLuc® as well as fusion combinations with mNeonGreen to shift the emission wavelength from blue to green. All BLING variants included the same Glt1 glutamate binding peptide (second domain), Igκ leader domain and PDGFRβ transmembrane domain. The additional BLING variants and their emission wavelengths are shown in Table 2 below.

TABLE 2

| luciferase | wavelength |
|---|---|
| Gaussia luciferase | ~480 |
| Renilla luciferase | ~535 |
| Nanoluc | ~460 |
| Nanoluc+mNeonGreen | ~520 |

### Example 5 — Dopamine-Binding Variants

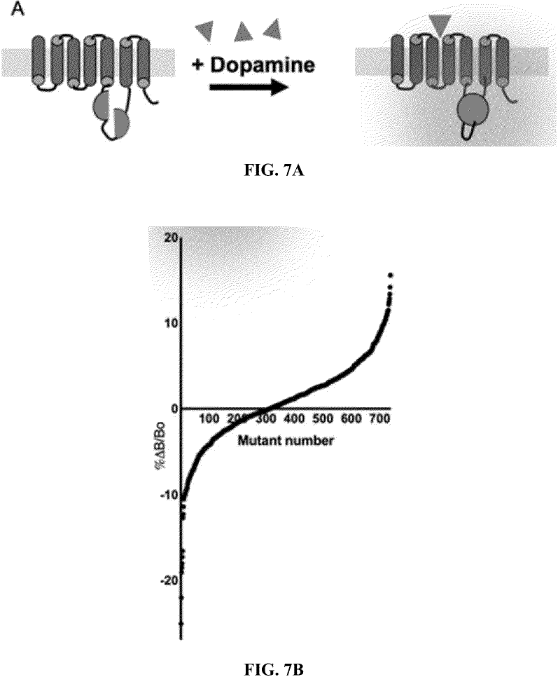

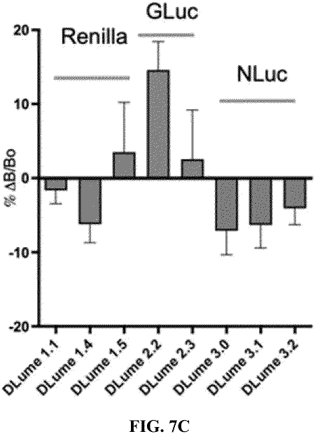

Bioluminescent indicators that luminesce in the presence of dopamine have also been prepared. To accomplish this, an initial library with circularly permutated luciferases with varied linker lengths and luciferases inserted between the S5 and S6 transmembrane domains of the human dopamine receptor were prepared to create bioluminescent dopamine indicators (DLume) (FIG. 7A). Various DLume variants with linker mutations were prepared and tested for luminescence in the presence of dopamine. (FIG. 7B) An improved DLume 3.2 though linker mutations and library screening to have approximately 20% increase in bioluminescence and it is the brightest DLume variant as also prepared. (FIG. 7C) These sensors and libraries were created using the same methodologies described above. The dopamine-detecting DLume variants are shown in Table 3 below.

TABLE 3

| Bioluminescent Indicator | Luciferase | Wavelength | SEQ ID NO (AA) | SEQ ID NO (DNA) |
|---|---|---|---|---|
| DLume 2.0 | Gaussia luciferase | ~480 | 12 | 15 |
| DLume 1.0 | Renilla luciferase | ~535 | 11 | 14 |
| DLume 3.0 | Nanoluc | ~460 | 13 | 16 |

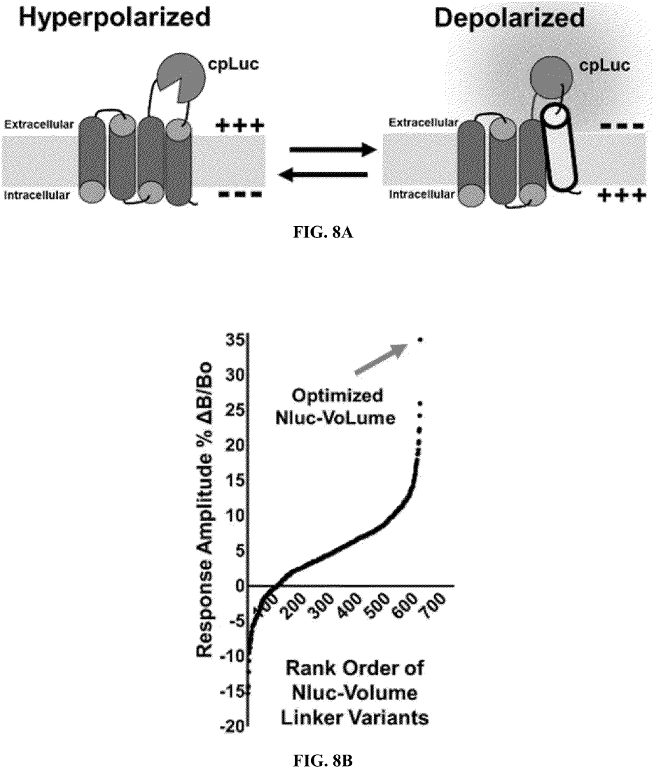

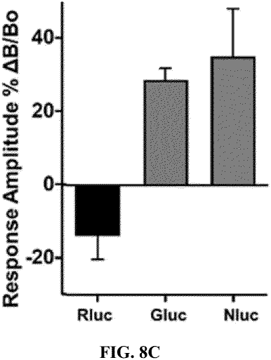

Additional bioluminescent indicators utilizing circularly permuted luciferases derived from the same species described above were also prepared. Unlike the glutamine- (BLING) and dopamine-(DLuma) detecting sensors above, these variants function independent of cofactors such as ATP and are inserted between the S3 and S4 transmembrane regions of luciferase (FIG. 8A) based on the voltage sensing domains used in the ASAP and Jedi voltage indicators utilizing different luciferases and linker compositions to create luminescent voltage indicators (VoLumes). The best VoLume has a 30% increase in luminescence upon depolarization when expressed in spiking HEK cells. (FIGS. 8B and 8C) The voltage-detecting variants comprising a voltage sensitive phosphatase in the second domain are shown in Table 4 below.

TABLE 4

| Bioluminescent Indicator | Luciferase | Wavelength | SEQ ID NO (AA) | SEQ ID NO (DNA) |
|---|---|---|---|---|
| VoLume 2.1 | Gaussia luciferase | ~480 | 18 | 21 |
| VoLume 1.0 | Renilla luciferase | ~535 | 17 | 20 |
| VoLume 3.0 | Nanoluc | ~460 | 19 | 22 |

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed:

1. A bioluminescent indicator comprising:
a first domain comprising a first portion of a bioluminescent peptide;
a second domain comprising a binding peptide which binds a ligand; and
a third domain comprising a second portion of the bioluminescent peptide,
wherein the bioluminescent protein indicator luminesces upon the ligand binding to the binding peptide.

2. The bioluminescent indicator of claim 1, wherein the bioluminescent peptide is luciferase.

3. The bioluminescent indicator of claim 2, wherein the luciferase is selected from the group consisting of luciferase from Gaussia princeps, luciferase from Renilla reniformis, or a synthetic luciferase.

4. The bioluminescent indicator of claim 1, wherein the binding peptide of the second domain binds to dopamine or glutamine.

5. The bioluminescent indicator of claim 1, further comprising a leader domain.

6. The bioluminescent indicator of claim 5, wherein the leader domain comprises a sequence comprising at least 85% sequence identity to an Igκ leader sequence.

7. The bioluminescent indicator of claim 1, further comprising a transmembrane domain.

8. The bioluminescent indicator of claim 7, wherein the transmembrane domain comprises a sequence comprising at least 85% sequence identity to PDGFRβ.

9. The bioluminescent indicator of claim 1, further comprising a first linker between the first domain and the second domain and a second linker between the second domain and the third domain.

10. The bioluminescent indicator of claim 1, having a sequence having at least 85% sequence homology to the sequence selected from the group consisting of SEQ ID NO:5, SEQ ID NO: 6, SEQ ID NO: 7, and SEQ ID NO: 8.

11. The bioluminescent indicator of claim 10, wherein the indicator has a sequence selected from the group consisting of SEQ ID NO: 5, SEQ ID NO: 6, SEQ ID NO: 7, and SEQ ID NO: 8.

12. The bioluminescent indicator of claim 1, which is encoded by a nucleotide sequence having at least 85% sequence identity the sequence selected from the group consisting of SEQ ID NO: 1, SEQ ID NO: 2, SEQ ID NO: 3, and SEQ ID NO: 4.

13. A composition comprising the bioluminescent indicator of claim 1 and a pharmaceutically acceptable excipient.

14. A nucleotide sequence comprising a DNA sequence encoding the bioluminescent indicator of claim 1.

15. The nucleotide sequence of claim 14, wherein the DNA sequence encoding the bioluminescent indicator has a sequence having at least 85% sequence identity with the sequence selected from the group consisting of SEQ ID NO: 1, SEQ ID NO: 2, SEQ ID NO: 3, and SEQ ID NO: 4.

16. The nucleotide sequence of claim 15, wherein the DNA sequence encoding the bioluminescent indicator comprises a sequence selected from the group consisting of SEQ ID NO: 1, SEQ ID NO: 2, SEQ ID NO: 3, and SEQ ID NO: 4.

17. A vector comprising the nucleotide sequence of claim 14.

18. The vector of claim 17, wherein the vector is an adeno-associated virus.

19. A method for detecting the presence of a ligand in a subject or sample comprising:
providing the bioluminescent indicator of claim 1 to the subject or sample; and
measuring luminescence of the bioluminescent indicator.

20. The method of claim 19, wherein the bioluminescent indicator is provided to the subject or sample by administration of a vector comprising an adeno-associated virus encoding the bioluminescent indicator.


SEQUENCE LISTING (22 sequences)

SEQ ID NO: 1 - DNA, 6991 bp, synthetic construct
  misc_feature 1..6991: note = BLING 0.1
  source 1..6991: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggaggaggcggtaccagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgacggatccggaggcggcggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 7012 bp, synthetic construct
  misc_feature 1..7012: note = BLING 0.2
  source 1..7012: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtggaggaggcggtaccagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgacggatccggaggcggcctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 7009 bp, synthetic construct
  misc_feature 1..7009: note = BLING 0.3
  source 1..7009: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggaggaggaggcggtaccagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgacggatccggaggcggcgtgaccggctggcggctgtgcgaacgcattctgctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 7003 bp, synthetic construct
  misc_feature 1..7003: note = BLING 1.0
  source 1..7003: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccatggagacagacacactcctgctatgggtactgctgctctgggttccaggttccactggtgacatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggttctccttctagtactctggacaaaatcgccaaaaacggtgtgattgtcgtcggtcaccgtgaatcttcagtgcctttctcttattacgacaatcagcaaaaagtggtgggttactcgcaggattactccaacgccattgttgaagcagtgaaaaagaaactcaacaaaccggacttgcaggtaaaactgattccgattacaacacaaaaccgtattccactgctgcaaaacggcactttcgattttgaatgtggttctaccaccaacaacgtcgaacgccaaaaacaggcggctttctctgacactattttcgtggtcggtacgcgcctgttgaccaaaaagggtggcgatatcaaagattttgccaacctgaaagacaaagccgtagtcgtcacttccggcactacctctgaagttttgctcaacaaactgaatgaagagcaaaaaatgaatatgcgcatcatcagcgccaaagatcacggtgactctttccgcaccctggaaagcggtcgtgccgttgcctttatgatggatgacgctctgctggccggtgaacgtgcgaaagcgaagaaaccagacaactgggaaatcgtcggcaagccgcagtctcaggaggcctacggttgtatgttgcgtaaagatgatccgcagttcaaaaagctgatggatgacaccatcgctcaggtgcagacctccggtgaagcggaaaaatggtttgataagtggttcaaaaatccaattccgccgaaaaacctgaacatgaatttcgaactgtcagacgaaatgaaagcactgttcaaagaaccgaatgaccctgctcctggcctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgctcgaggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttagtaacatatgctctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - protein, 514 aa, synthetic construct
MGVKVLFALICIAVAEAKPTENNEDFNIVAVASNFATTDLDADRGKLPGKKLPLEVLKELEANARKAGCTRGCLICLSHIKCTPKMKKFIPGRCHTYEGDKESAQGGGGTSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDGSGGGGGIGEAIVDIPEIPGFKDLEPLEQFIAQVDLCVDCTTGCLKGLANVQCSDLLKKWLPQRCATFASKIQGQVDKIKGAGGDLEAVGQDTQEVIVVPHSLPFKVVVISAILALVVLTIISLIILIMLWQKKPR

SEQ ID NO: 6 - protein, 521 aa, synthetic construct
METDTLLLWVLLLWVPGSTGDMVFTLEDFVGDWRQTAGYNLDQVLEQGGVSSLFQNLGVSVTPIQRIVLSGENGLKIDIHVIIPYEGGGGGTSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDGSGGGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILALEAVGQDTQEVIVVPHSLPFKVVVISAILALVVLTIISLIILIMLWQKKPR

SEQ ID NO: 7 - protein, 520 aa, synthetic construct
METDTLLLWVLLLWVPGSTGDMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGGGGGTSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDGSGGGVTGWRLCERILLEAVGQDTQEVIVVPHSLPFKVVVISAILALVVLTIISLIILIMLWQKKPR

SEQ ID NO: 8 - protein, 518 aa, synthetic construct
METDTLLLWVLLLWVPGSTGDMVFTLEDFVGDWRQTAGYNLDQVLEQGGVSSLFQNLGVSVTPIQRIVLSGENGLKIDIHVIIPYEGSPSSTLDKIAKNGVIVVGHRESSVPFSYYDNQQKVVGYSQDYSNAIVEAVKKKLNKPDLQVKLIPITTQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDPAPGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILALEAVGQDTQEVIVVPHSLPFKVVVISAILALVVLTIISLIILIMLWQKKPR

SEQ ID NO: 9 - protein, 5 aa, synthetic construct
GGGGS

SEQ ID NO: 10 - protein, 5 aa, synthetic construct
PAPAP

SEQ ID NO: 11 - protein, 750 aa, synthetic construct
MKTIIALSYIFCLVFADYKDDDDAMRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKLSSLIKPDVVQIVRNYNAYLRASDDLPKLFIESDPGFFSNAIVEGAKKFPNTEFVKVKGLHFLQEDAPDEMGKYIKSFVERVLKNEQMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGNHDQLKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 12 - protein, 624 aa, synthetic construct
MKTIIALSYIFCLVFADYKDDDDAMRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKGGIGEAIVDIPEIPGFKDLEPLEQFIAQVDLCVDCTTGCLKGLANVQCSDLLKKWLPQRCATFASKIQGQVDKIKGAGGDGGSGGSTGGSMGVKVLFALICIAVAEAKPTENNEDFNIVAVASNFATTDLDADRGKLPGKKLPLEVLKELEANARKAGCTRGCLICLSHIKCTPKMKKFIPGRCHTYEGDKESAQKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 13 - protein, 620 aa, synthetic construct
MKTIIALSYIFCLVFADYKDDDDAMRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKLSSLILSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGSTGGSMVFTLEDFVGDWRQTAGYNLDQVLEQGGVSSLFQNLGVSVTPIQRIVLSGENGLKIDIHVIIPYEGNHDQLKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 14 - DNA, 2250 bp, synthetic construct
  source 1..2250: mol_type = other DNA
atgaagacgatcatcgccctgagctacatcttctgcctggtgttcgccgactacaaggacgatgatgacgccatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaatcagcttttacatccctgtggccatcatgattgtcacctacaccaggatctacaggattgctcagaaactgagctcactcattaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactgggggagcgctctggcctttcactacgcctacgagcaccaagacaggatcaaggccatcgtccatatggagagtgtcgtggacgtgatcgagtcctggatggggtggcctgacatcgaggaggagctggccctgatcaagagcgaagagggcgagaaaatggtgcttgagaataacttcttcgtcgagaccctgttgccaagcaagatcatgcggaaactggagcctgaggagttcgctgcctacctggagccattcaaggagaagggcgaggttagacggcctaccctctcctggcctcgcgagatccctctcgttaagggaggcaatcatgaccaactgaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacc

SEQ ID NO: 15 - DNA, 1872 bp, synthetic construct
  source 1..1872: mol_type = other DNA
atgaagacgatcatcgccctgagctacatcttctgcctggtgttcgccgactacaaggacgatgatgacgccatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaatcagcttttacatccctgtggccatcatgattgtcacctacaccaggatctacaggattgctcagaaaggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacggtggctctggaggttctaccggcggtagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacc

SEQ ID NO: 16 - DNA, 1860 bp, synthetic construct
  source 1..1860: mol_type = other DNA
atgaagacgatcatcgccctgagctacatcttctgcctggtgttcgccgactacaaggacgatgatgacgccatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaatcagcttttacatccctgtggccatcatgattgtcacctacaccaggatctacaggattgctcagaaactgagctcactcattctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggtggctctggtggatctaccggcggtagcatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtaatcatgaccaactgaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacc

SEQ ID NO: 17 - protein, 500 aa, synthetic construct
METTVRYEQGSELTKTSSSPTADEPTIKIDDGRDEGNEQDSCSNTIRRKISPFVMSFGFRVFGVVLIIVDIIVVIVDLAISEKKRGIREILEGVSLAIALFFLVDVLMRVFVEGFKNYFRSKLVTLDAVIVVGTLLINMTYSFSDLAAFNSEKPDVVQIVRNYNAYLRASDDLPKLFIESDPGFFSNAIVEGAKKFPNTEFVKVKGLHFLQEDAPDEMGKYIKSFVERVLKNEQMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGNTDQMPHMVTLLRVLKIVILIRIFRLASQKKQLEVVT

SEQ ID NO: 18 - protein, 383 aa, synthetic construct
METTVRYEQGSELTKTSSSPTADEPTIKIDDGRDEGNEQDSCSNTIRRKISPFVMSFGFRVFGVVLIIVDIIVVIVDLAISEKKRGIREILEGVSLAIALFFLVDVLMRVFVEGFKNYFRSKLVTLDAVIVVGTLLINMTYSFSDLAAFNSEGGIGEAIVDIPEIPGFKDLEPLEQFIAQVDLCVDCTTGCLKGLANVQCSDLLKKWLPQRCATFASKIQGQVDKIKGAGGDGGSGGSTGGSMGVKVLFALICIAVAEAKPTENNEDFNIVAVASNFATTDLDADRGKLPGKKLPLEVLKELEANARKAGCTRGCLICLSHIKCTPKMKKFIPGRCHTYEGDKESAQTDQMPHMVTLLRVLKIVILIRIFRLASQKKQLEVVT

SEQ ID NO: 19 - protein, 371 aa, synthetic construct
METTVRYEQGSELTKTSSSPTADEPTIKIDDGRDEGNEQDSCSNTIRRKISPFVMSFGFRVFGVVLIIVDIIVVIVDLAISEKKRGIREILEGVSLAIALFFLVDVLMRVFVEGFKNYFRSKLVTLDAVIVVGTLLINMTYSFSDLAAFNSEXXLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGSTGGSMVFTLEDFVGDWRQTAGYNLDQVLEQGGVSSLFQNLGVSVTPIQRIVLSGENGLKIDIHVIIPYEGXXQMPHMVTLLRVLKIVILIRIFRLASQKKQLEVVT

SEQ ID NO: 20 - DNA, 1500 bp, synthetic construct
  source 1..1500: mol_type = other DNA
atggagacgactgtgaggtatgaacaggggtcagagctcactaaaacttcgagctctccaacagcagatgagcccacgataaagattgatgatggtcgtgatgagggtaatgaacaagacagctgttccaataccattaggagaaaaatttccccgtttgtgatgtcatttggattcagagtatttggagttgtgcttatcattgtagacatcatagtggtgattgtggatctggccatcagtgagaagaaaagaggcattagagagattcttgaaggtgtttccctggctatagcactcttcttccttgttgatgttctcatgagagtgtttgttgaaggcttcaagaactatttccggtccaaactggttactttggatgcagtcatagtagtgggcactctgctaattaatatgacctactccttctctgaccttgctgcctttaacagcgagaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactgggggagcgctctggcctttcactacgcctacgagcaccaagacaggatcaaggccatcgtccatatggagagtgtcgtggacgtgatcgagtcctggatggggtggcctgacatcgaggaggagctggccctgatcaagagcgaagagggcgagaaaatggtgcttgagaataacttcttcgtcgagaccctgttgccaagcaagatcatgcggaaactggagcctgaggagttcgctgcctacctggagccattcaaggagaagggcgaggttagacggcctaccctctcctggcctcgcgagatccctctcgttaagggaggcaatacagatcagatgccgcacatggttactcttcttcgagttctgaaaattgttatcttaataagaatatttcgcctggcttcacagaagaaacaacttgaagtggtaacc

SEQ ID NO: 21 - DNA, 1149 bp, synthetic construct
  source 1..1149: mol_type = other DNA
atggagacgactgtgaggtatgaacaggggtcagagctcactaaaacttcgagctctccaacagcagatgagcccacgataaagattgatgatggtcgtgatgagggtaatgaacaagacagctgttccaataccattaggagaaaaatttccccgtttgtgatgtcatttggattcagagtatttggagttgtgcttatcattgtagacatcatagtggtgattgtggatctggccatcagtgagaagaaaagaggcattagagagattcttgaaggtgtttccctggctatagcactcttcttccttgttgatgttctcatgagagtgtttgttgaaggcttcaagaactatttccggtccaaactggttactttggatgcagtcatagtagtgggcactctgctaattaatatgacctactccttctctgaccttgctgcctttaacagcgagggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacggtggctctggaggttctaccggcggtagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagacagatcagatgccgcacatggttactcttcttcgagttctgaaaattgttatcttaataagaatatttcgcctggcttcacagaagaaacaacttgaagtggtaacc

SEQ ID NO: 22 - DNA, 1113 bp, synthetic construct
  source 1..1113: mol_type = other DNA
atggagacgactgtgaggtatgaacaggggtcagagctcactaaaacttcgagctctccaacagcagatgagcccacgataaagattgatgatggtcgtgatgagggtaatgaacaagacagctgttccaataccattaggagaaaaatttccccgtttgtgatgtcatttggattcagagtatttggagttgtgcttatcattgtagacatcatagtggtgattgtggatctggccatcagtgagaagaaaagaggcattagagagattcttgaaggtgtttccctggctatagcactcttcttccttgttgatgttctcatgagagtgtttgttgaaggcttcaagaactatttccggtccaaactggttactttggatgcagtcatagtagtgggcactctgctaattaatatgacctactccttctctgaccttgctgcctttaacagcgagnnknnkctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggtggctctggtggatctaccggcggtagcatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtnnknnkcagatgccgcacatggttactcttcttcgagttctgaaaattgttatcttaataagaatatttcgcctggcttcacagaagaaacaacttgaagtggtaacc